(19) United States
(12) Patent Application Publication
(10) Pub. No.: US 2018/0057892 A1
(43) Pub. Date: Mar. 1, 2018

(54) PRIMER PAIR, KIT AND METHOD OF DETECTING BABESIA CANIS

(71) Applicant: DELTA ELECTRONICS INT'L (SINGAPORE) PTE LTD, Singapore (SG)

(72) Inventors: Yong Zhang, Singapore (SG); Chih-Yu Chao, Singapore (SG); Wanyue Li, Singapore (SG); Ying-Ting Chen, Singapore (SG)

(21) Appl. No.: 15/259,239

(22) Filed: Sep. 8, 2016

(30) Foreign Application Priority Data
Aug. 25, 2016 (SG) 10201607084X

(51) Int. Cl. C12Q 1/68

(52) U.S. Cl. CPC C12Q 1/6893

(57) ABSTRACT

Primer pair, kit and method of detecting Babesia canis are disclosed. The primer pair includes a forward primer and a reverse primer, and the kit includes the primer pair and a probe. The forward primer has a sequence of SEQ ID NO: 1, the reverse primer has a sequence of SEQ ID NO: 2, and the probe has a sequence of SEQ ID NO: 3.

### FIELD OF THE INVENTION

The present invention relates to primer pair, kit and method of detecting Babesia canis, and more particularly to primer pair, kit and method of detecting Babesia canis.

### BACKGROUND OF THE INVENTION

Various kinds of Babesia spp. can infect dogs, and the most common ones are Babesia canis (also called large Babesia) and Babesia gibsoni (also called small Babesia). Babesia canis is a protozoal parasite which infects red blood cells of dogs and can lead to anemia. Babesia canis is transmitted by the brown dog tick (Rhipicephalus sanguineus) and is one of the most common piroplasm infections. The brown dog tick is adapted to warmer climate, therefore most infections come from South Asia, Southeast Asia, Japan, South Korea, Central East of China, Oceania, Europe and United States.

Since the therapeutic approaches for Babesia canis and Babesia gibsoni are different, it is important to rapidly and correctly diagnose Babesia canis to avoid inadequate treatment. However, Babesia canis is not easy to diagnose. Generally, the methods employed for Babesia canis diagnosis includes blood smear, serologic diagnosis and molecular diagnosis, but each method has some limitations.

A veterinarian can perform direct pathogen detection in blood smears stained by Giemsa, but it is hard to differentiate large and small Babesia species in stained blood smears and this method significantly depends for its accuracy on well-trained and experienced technologists. Besides, this method requires fresh samples to preserve organism viability and morphology, and thus the samples must be processed very quickly.

Serologic diagnosis may be helpful in identifying the presence of antibodies to Babesia canis, but serology cannot distinguish between animals with an acute or chronic infection. The limitations of serologic diagnosis are cross-reactions (especially between different Babesia species), which results in reduced specificity, and false-negative findings in young or immunosuppressed dogs, or early in the course of infection before seroconversion has occurred.

The most current and best way to diagnose Babesia canis is molecular diagnosis, especially by polymerase chain reaction (PCR) testing. PCR, which is more sensitive and specific technique, offers an alternative approach for the diagnosis of babesiosis. An 18S rRNA gene sequence has been helpful in identifying species of Babesia and related protozoa. For example, the canine babesiosis 18S ribosomal RNA (18S) gene genesig standard kit provided by Primerdesign Ltd is used to diagnose canine babesiosis. However, this kit cannot differentiate Babesia gibsoni (small Babesia) and Babesia canis (large Babesia).

Thus, there is a need of providing a method of specifically detecting Babesia canis in order to select an appropriate treatment.

### SUMMARY OF THE INVENTION

An object of the present invention is to provide a primer pair of detecting Babesia canis in order to select an appropriate treatment.

An another object of the present invention is to provide a kit of detecting Babesia canis in order to select an appropriate treatment.

An additional object of the present invention is to provide a method of detecting Babesia canis in order to select an appropriate treatment.

According to an aspect of the present invention, there is provided a primer pair of detecting Babesia canis, comprising a forward primer having a sequence of 5′-TAGTTTGAAACCCGCCTT-3′ and a reverse primer having a sequence of 5′-GATGGGTCAGAAACTTGAA-3′. The forward primer and the reverse primer are used for real-time polymerase chain reaction.

According to another aspect of the present invention, there is provided a kit of detecting Babesia canis, comprising a forward primer having a sequence of 5′-TAGTTTGAAACCCGCCTT-3′, a reverse primer having a sequence of 5′-GATGGGTCAGAAACTTGAA-3′ and a probe having a sequence of 5′-CATCGCTAAATGCGATTCGCCA-3′. The forward primer, the reverse primer and the probe are used for real-time polymerase chain reaction. The probe is labeled with a 5′-reporter dye and a 3′-quencher.

According to an additional aspect of the present invention, there is provided a method of detecting Babesia canis, the method comprising amplifying nucleic acid from Babesia canis using real-time polymerase chain reaction with a forward primer having a sequence of 5′-TAGTTTGAAACCCGCCTT-3′, a reverse primer having a sequence of 5′-GATGGGTCAGAAACTTGAA-3′ and a probe having a sequence of 5′-CATCGCTAAATGCGATTCGCCA-3′. The probe is labeled with a 5′-reporter dye and a 3′-quencher.

According to an additional aspect of the present invention, there is provided a kit of detecting Babesia canis, comprising a forward primer having a sequence of 5′-TAGTTTGAAACCCGCCTT-3′, a reverse primer having a sequence of 5′-GATGGGTCAGAAACTTGAA-3′ and a probe having a sequence of 5′-TGGCGAATCGCATTTAGCGATG-3′.

According to an additional aspect of the present invention, there is provided a method of detecting Babesia canis, the method comprising amplifying nucleic acid from Babesia canis using real-time polymerase chain reaction with a forward primer having a sequence of 5′-TAGTTTGAAACCCGCCTT-3′, a reverse primer having a sequence of 5′-GATGGGTCAGAAACTTGAA-3′ and a probe having a sequence of 5′-TGGCGAATCGCATTTAGCGATG-3′.

The above objects and advantages of the present invention become more readily apparent to those ordinarily skilled in the art after reviewing the following detailed description and accompanying drawings, in which:

### BRIEF DESCRIPTION OF THE DRAWINGS

FIG. 1 shows the corresponding positions of the forward primer, the reverse primer and the probe on the sequence of the 18S rRNA gene;

FIG. 2 shows the DNA sequences of the forward primer, the reverse primer and the probe;

FIG. 3 shows the sequence alignment of 18S rRNA from several Babesia species; and

FIGS. 4A and 4B show the analysis of the amplification of the real-time PCR assay.

### DETAILED DESCRIPTION OF THE PREFERRED EMBODIMENT

The present invention will now be described more specifically with reference to the following embodiments. It is to be noted that the following descriptions of preferred embodiments of this invention are presented herein for purpose of illustration and description only; it is not intended to be exhaustive or to be limited to the precise form disclosed.

The present invention utilizes real-time polymerase chain reaction (Real-time PCR), also called quantitative polymerase chain reaction (Q-PCR), with probe-based detection for detecting Babesia canis. In Real-time PCR, the specific forward and reverse primers and probe hybridize to the DNA target of Babesia canis, wherein the probe is labeled with a 5′-reporter dye and a 3′-quencher. During PCR amplification, the probe is cleaved and the reporter dye and quencher are separated, so that the resulting increase in fluorescence can be detected. In an embodiment, the reporter dye is FAM fluorescence, and the quencher is BHQ1 group.

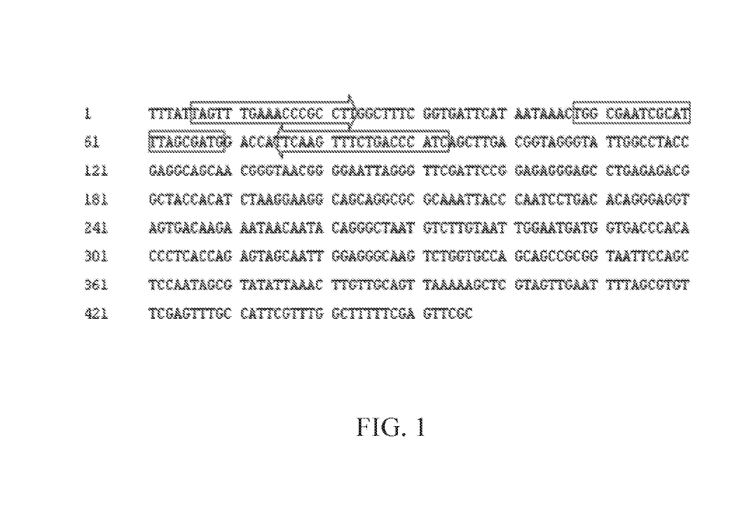

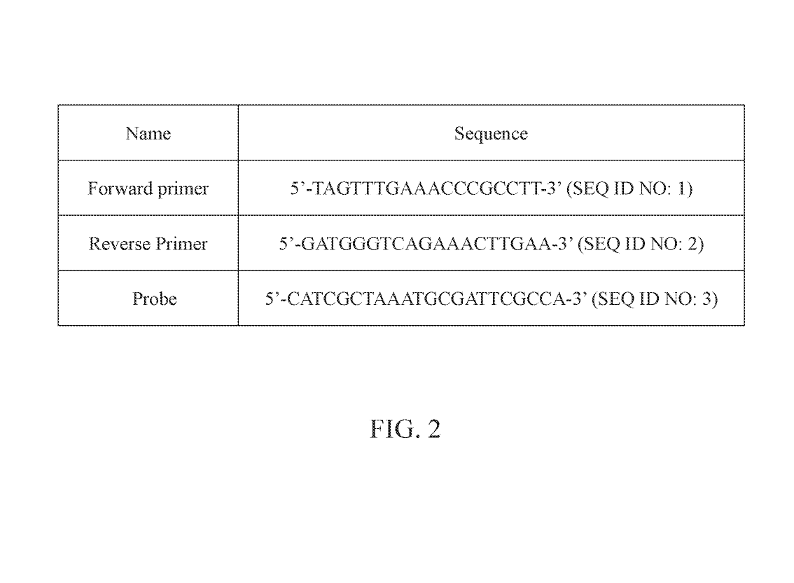

The DNA target for this assay is a variable region of the 18S rRNA gene (GenBank accession number: KP896299.1) that contains sequence that is species-specific for Babesia canis. PCR primers and probe are designed using Primer3 and chosen on the basis of GC content and lack of hairpin structures. FIG. 1 shows the corresponding positions of the forward primer, the reverse primer and the probe on the sequence of the 18S rRNA gene. As shown in FIG. 1, the forward primer starts at position 6, the probe starts at position 69, and the reverse primer starts at position 93. This primers and probe combination is predicted to amplify the DNA of Babesia canis strains with an amplicon size of 88-bp. FIG. 2 shows the DNA sequences of the forward primer, the reverse primer and the probe, wherein the forward primer (SEQ ID NO: 1) includes 18-mer, the reverse primer (SEQ ID NO: 2) includes 19-mer, and the probe (SEQ ID NO: 3) includes 22-mer.

To ascertain the specificity of the PCR primers and probe for Babesia canis, the primer pair, including the forward primer and the reverse primer, and the probe are checked by Primer-BLAST from NCBI, and the blast result shows that no other similar species have 100% same fragment compare to the primer pairs and the probe of the present invention.

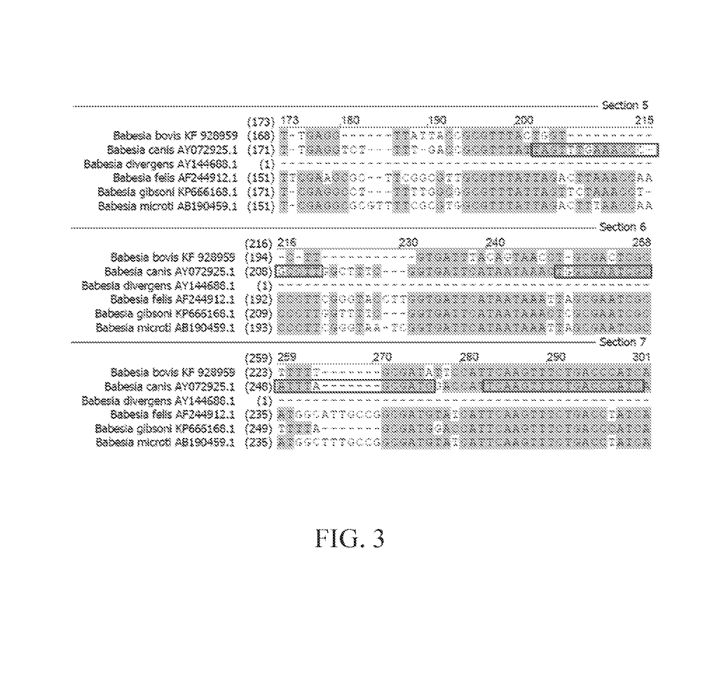

Moreover, to get more information about the specificity of the primer pairs and the probe of the present invention, DNA alignment tool is used to analyze other similar DNA fragments. FIG. 3 shows the sequence alignment of 18S rRNA from several Babesia species. For the Babesia canis sequence, the forward primer is located at the 195^{th} base pair to 212^{th} base pair, the probe is located at the 237^{th} base pair to 258^{th} base pair, and the reverse primer is located at the 264^{th} base pair to 282^{th} base pair. In these fragments, there is no exactly the same sequence in other similar species. The result demonstrates that the specificity of the primer pair and the probe is quite high, and the primer pair and the probe can only used to amplify and detect the 18S rRNA gene of Babesia canis.

Therefore, the present invention provides a primer pair of detecting Babesia canis, comprising a forward primer having a sequence of 5′-TAGTTTGAAACCCGCCTT-3′ and a reverse primer having a sequence of 5′-GATGGGTCAGAAACTTGAA-3′. The present invention also provides a kit of detecting Babesia canis, comprising a forward primer having a sequence of 5′-TAGTTTGAAACCCGCCTT-3′, a reverse primer having a sequence of 5′-GATGGGTCAGAAACTTGAA-3′ and a probe having a sequence of 5′-CATCGCTAAATGCGATTCGCCA-3′. On the other hand, the present invention also provides a method of detecting Babesia canis, the method comprising amplifying nucleic acid from Babesia canis using real-time polymerase chain reaction with a forward primer having a sequence of 5′-TAGTTTGAAACCCGCCTT-3′, a reverse primer having a sequence of 5′-GATGGGTCAGAAACTTGAA-3′ and a probe having a sequence of 5′-CATCGCTAAATGCGATTCGCCA-3′.

In some other embodiments, since the primer pair of the present invention is specific to Babesia canis, all the sequence located between the forward primer and the reverse primer may be used as the probe sequence, and thus, the probe sequence is not limited to the aforesaid sequence. Further, the probe can be designed to hybridize to any strand of the DNA, so both the complementary sequences at the same location can be used as the probe sequence. For example, the complementary sequence 5′-TGGCGAATCGCATTTAGCGATG-3′ (SEQ ID NO: 4) of the aforesaid probe sequence can be used as the probe sequence.

Therefore, the present invention also provides a kit of detecting Babesia canis, comprising a forward primer having a sequence of 5′-TAGTTTGAAACCCGCCTT-3′, a reverse primer having a sequence of 5′-GATGGGTCAGAAACTTGAA-3′ and a probe having a sequence of 5′-TGGCGAATCGCATTTAGCGATG-3′. On the other hand, the present invention also provides a method of detecting Babesia canis, the method comprising amplifying nucleic acid from Babesia canis using real-time polymerase chain reaction with a forward primer having a sequence of 5′-TAGTTTGAAACCCGCCTT-3′, a reverse primer having a sequence of 5′-GATGGGTCAGAAACTTGAA-3′ and a probe having a sequence of 5′-TGGCGAATCGCATTTAGCGATG-3′.

The following describes an example of the method of detecting Babesia canis of the present invention.

First, DNA is extracted from 200 μl of EDTA-preserved whole blood using the QIAamp DNA blood Mini kit for blood protocol (Qiagen) and eluted in 100 μl of elution buffer. Then the real-time PCR assay is performed on the Bio-Rad real-time PCR machine (CFX96). The PCR reaction mixture includes 10 μl of KAPA Fast probe universal master mix, 250 nM of forward and reverse primers and 250 nM of probe, wherein the forward primer has a sequence of 5′-TAGTTTGAAACCCGCCTT-3′, the reverse primer has a sequence of 5′-GATGGGTCAGAAACTTGAA-3′ and the probe has a sequence of 5′-CATCGCTAAATGCGATTCGCCA-3′. 3 μl extracted DNA template is added to each reaction in a total volume of 20 μl. Cycling conditions are as follows: 95° C. for 3 min, followed by 40 cycles of denaturation at 95° C. for 3 sec, and annealing/extension at 60° C. for 20 sec.

A Babesia canis-positive control is constructed by cloning the 456-bp 18S rRNA gene fragment into a vector (RBC Cloning System). A series of seven 10-fold dilutions are prepared from this recombinant plasmid DNA (1.25×10, 1.25×10², 1.25×10³, 1.25×10⁴, 1.25×10⁵, 1.25×10⁶, 1.25×10⁸ copies/μl). The dilution series are analyzed in duplicate to determine the lower limit of Babesia canis DNA detection and the linearity and efficiency of amplification of this real-time PCR assay.

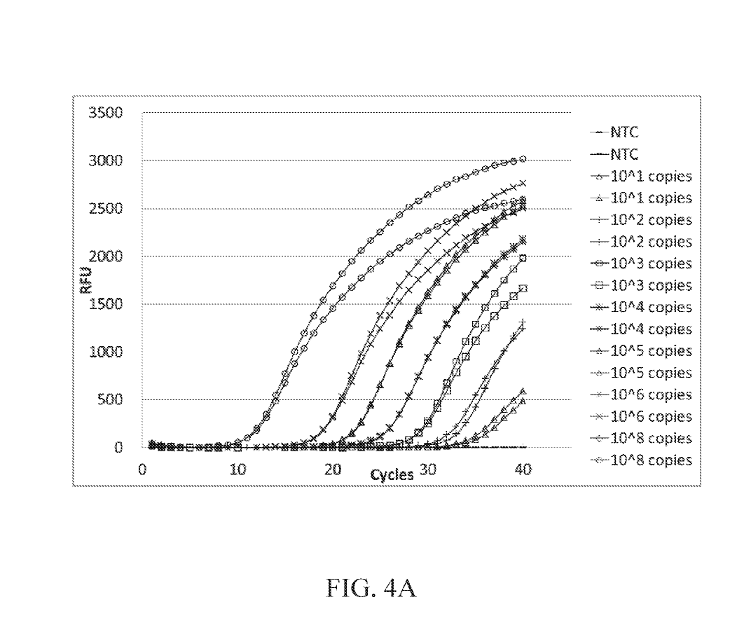

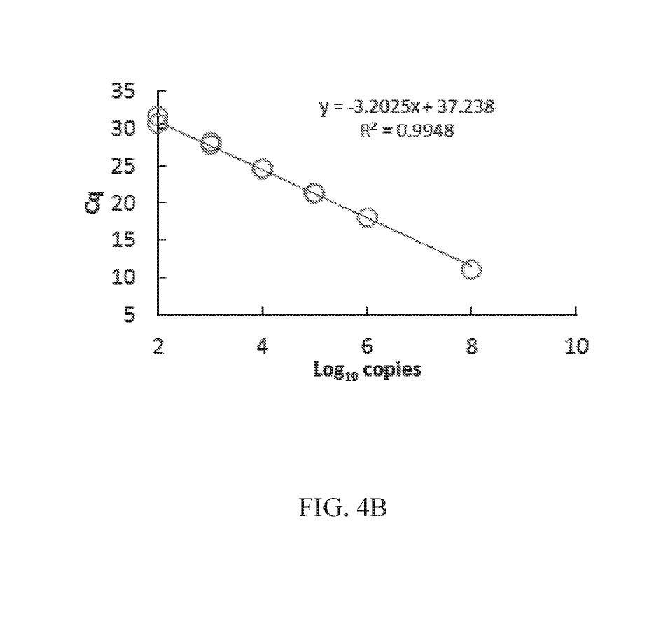

FIGS. 4A and 4B show the analysis of the amplification of the real-time PCR assay. FIG. 4A shows the amplification curve of different copies of plasmid samples, which reveals that the assay has high sensitivity. FIG. 4B show the assay has good linearity, and thus, the assay could be expanded as a quantitative assay to estimate gene copy number and, by extension, percent parasitemia in clinical samples.

In conclusion, the present invention provides a method of detecting Babesia canis using real-time PCR with specific primer pairs and probe. The method of the present invention has advantage of high sensitivity, and should allow the detection of low parasitemia in subclinically infected cases. The method of the present invention further has advantage of high specificity, which is able to differentiate Babesia species, and this is important in order to select an appropriate treatment for Babesia canis, as large Babesia and small Babesia require different therapeutic approaches.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A primer pair of detecting Babesia canis, comprising a forward primer having a sequence of 5′-TAGTTTGAAACCCGCCTT-3′ and a reverse primer having a sequence of 5′-GATGGGTCAGAAACTTGAA-3′.

2. The primer pair according to claim 1 wherein the forward primer and the reverse primer are used for real-time polymerase chain reaction.

3. A kit of detecting Babesia canis, comprising a forward primer having a sequence of 5′-TAGTTTGAAACCCGCCTT-3′, a reverse primer having a sequence of 5′-GATGGGTCAGAAACTTGAA-3′ and a probe having a sequence of 5′-CATCGCTAAATGCGATTCGCCA-3′.

4. The kit according to claim 3 wherein the forward primer, the reverse primer and the probe are used for real-time polymerase chain reaction.

5. The kit according to claim 3 wherein the probe is labeled with a 5′-reporter dye and a 3′-quencher.

6. A method of detecting Babesia canis, the method comprising amplifying nucleic acid from Babesia canis using real-time polymerase chain reaction with a forward primer having a sequence of 5′-TAGTTTGAAACCCGCCTT-3′, a reverse primer having a sequence of 5′-GATGGGTCAGAAACTTGAA-3′ and a probe having a sequence of 5′-CATCGCTAAATGCGATTCGCCA-3′.

7. The method according to claim 6 wherein the probe is labeled with a 5′-reporter dye and a 3′-quencher.

8. A kit of detecting Babesia canis, comprising a forward primer having a sequence of 5′-TAGTTTGAAACCCGCCTT-3′, a reverse primer having a sequence of 5′-GATGGGTCAGAAACTTGAA-3′ and a probe having a sequence of 5′-TGGCGAATCGCATTTAGCGATG-3′.

9. A method of detecting Babesia canis, the method comprising amplifying nucleic acid from Babesia canis using real-time polymerase chain reaction with a forward primer having a sequence of 5′-TAGTTTGAAACCCGCCTT-3′, a reverse primer having a sequence of 5′-GATGGGTCAGAAACTTGAA-3′ and a probe having a sequence of 5′-TGGCGAATCGCATTTAGCGATG-3′.


SEQUENCE LISTING (4 sequences)

SEQ ID NO: 1 - DNA, 18 bp, Artificial Sequence
  Synthetically generated primer
tagtttgaaacccgcctt

SEQ ID NO: 2 - DNA, 19 bp, Artificial Sequence
  Synthetically generated primer
gatgggtcagaaacttgaa

SEQ ID NO: 3 - DNA, 22 bp, Artificial Sequence
  Synthetically generated probe
catcgctaaatgcgattcgcca

SEQ ID NO: 4 - DNA, 22 bp, Artificial Sequence
  Synthetically generated probe
tggcgaatcgcatttagcgatg